(19) United States
(12) Patent Application Publication
(10) Pub. No.: US 2020/0289000 A1
(43) Pub. Date: Sep. 17, 2020

(54) TOILET WITH VASCULAR HEALTH REPORTING

(71) Applicant: Hall Labs, LLC, Provo, UT (US)

(72) Inventors: David R. Hall, Provo, UT (US); Joshua Larsen, Spanish Fork, UT (US); Jared Reynolds, Spanish Fork, UT (US); Kevin Jeffrey Campbell, Spanish Fork, UT (US); Travis Niederhauser, Mapleton, UT (US); Daniel Hendricks, Provo, UT (US)

(21) Appl. No.: 16/818,900

(22) Filed: Mar. 13, 2020

Related U.S. Application Data
(60) Provisional application No. 62/817,758, filed on Mar. 13, 2019.

(51) Int. Cl. A61B 5/024; A61B 5/0205; A61B 5/11; G01N 33/493; G16H 10/60; G16H 15/00; G16H 10/40

(52) U.S. Cl. CPC A61B 5/02427; A61B 5/0205; A61B 5/1102; A47K 13/105; G16H 10/60; G16H 15/00; G16H 10/40; G01N 33/493

(57) ABSTRACT

A system for providing a report on vascular health of a user is disclosed. The system includes a toilet with a bowl adapted to receive excreta from the user and a processor. The toilet includes a seat with weight and PPG sensors. BCG data is derived from the weight sensor data. PPG data is derived from the PPG sensor. The processor performs a comparison of the data from the weight sensor and the data from the PPG sensor and preexisting data in a database. The processor then generates a report on the user's vascular health based on the comparison.

### CROSS-REFERENCE TO RELATED APPLICATIONS

This application is a continuation-in-part of U.S. Provisional Application No. 62/817,758, entitled MEDICAL TOILET FOR COLLECTING CARDIOVASCULAR AND PERIPHERAL VASCULAR MEASUREMENTS, filed on 13 Mar. 2019, the entire contents of which are hereby incorporated herein by reference in its entirety.

### TECHNICAL FIELD

The present disclosure relates to smart toilets. More particularly, it relates to diagnostic medical toilets equipped to provide health and wellness information to the user.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

Just a few examples of smart toilets and other bathroom devices can be seen in the following U.S. patents and Published applications: U.S. Pat. No. 9,867,513, entitled “Medical Toilet With User Authentication”; U.S. Pat. No. 10,123,784, entitled “In Situ Specimen Collection Receptacle In A Toilet And Being In Communication With A Spectral Analyzer”; U.S. Pat. No. 10,273,674, entitled “Toilet Bowl For Separating Fecal Matter And Urine For Collection And Analysis”; US 2016/0000378, entitled “Human Health Property Monitoring System”; US 2018/0020984, entitled “Method Of Monitoring Health While Using A Toilet”; US 2018/0055488, entitled “Toilet Volatile Organic Compound Analysis System For Urine”; US 2018/0078191, entitled “Medical Toilet For Collecting And Analyzing Multiple Metrics”; US 2018/0140284, entitled “Medical Toilet With User Customized Health Metric Validation System”; US 2018/0165417, entitled “Bathroom Telemedicine Station.” The disclosures of all these patents and applications are incorporated by reference in their entireties.

### SUMMARY

In a first aspect, the disclosure provides a system for providing a report on vascular health of a user is disclosed. The system includes a toilet with a bowl adapted to receive excreta from the user and a processor. The toilet includes a seat with weight and PPG sensors. BCG data is derived from the weight sensor data. PPG data is derived from the PPG sensor. The processor performs a comparison of the data from the weight sensor and the data from the PPG sensor and preexisting data in a database. The processor then generates a report on the user's vascular health based on the comparison.

In a second aspect, the disclosure provides additional information about how the data can be generated, used, or augmented through the use of additional sensors.

Further aspects and embodiments are provided in the foregoing drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is an isometric view of the toilet according to one embodiment of the invention.

FIG. 2 is a top view of the toilet of FIG. 1.

FIG. 3 is a view of the bottom of the seat and lid of the toilet of FIG. 1.

FIG. 4 is a view from the side of the toilet of FIG. 1 with the cover removed.

FIG. 5 is a view showing the top of a foot scale used in one embodiment of the invention.

FIG. 6 is a view of the bottom of the foot scale of FIG. 5.

FIG. 7 is an isometric view of a toilet used in another embodiment of the invention.

FIG. 8 is a top view of the toilet of FIG. 7.

FIG. 9 is a view of the bottom of the seat of the toilet of FIG. 7.

FIG. 10 is a partial view of the toilet of FIG. 7 with the cover removed.

FIG. 11 is a detail view of a handle used in an embodiment of the invention.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, the term “excreta” refers to any substance released from the body including urine, feces, menstrual discharge, and anything contained or excreted therewith.

As used herein, “toilet” is meant to refer to any device or system for receiving human excreta, including urinals.

As used herein, the term “bowl” refers to the portion of a toilet that is designed to receive excreta.

As used herein, the term “base” refers to the portion of the toilet below and around the bowl supporting it.

As used herein, the term “user” refers to any individual who interacts with the toilet and deposits excreta therein.

As used herein, “vascular” is meant to refer to aspects of the body that relate to the body's circulatory system, including the cardiovascular system and lymphatic system.

As used herein, “peripheral vascular” is meant to refer parts of the vascular system not in the chest or abdomen, such as arteries and veins in the arms, hands, legs, and feet.

As used herein, “health and wellness” is meant to refer a person's mental and physical condition. It is a comprehensive term that encompasses physical conditions such as fitness, effectiveness of normal biological processes, abnormalities of biological processes, effects of foreign organisms and chemicals influencing the body, and effects of other environmental stressors like events that trigger an emotional or physiological response. Additionally, it encompasses mental state, especially as affected by a person's physical condition.

As used herein in association with analyzing user data, the term “neural net” is meant to refer to a type of computer analysis that compares the user's current data with preexisting data through algorithms incorporating “deep learning”. Deep learning includes presenting the analysis algorithm with sample data and health states or conditions associated with that data; generally, the larger the data samples, the better results. Based on the sample data and its associated characteristics, the neural net analyzes the user data to find relationships or correlations between characteristics of the data and the potential states and conditions.

### Exemplary Embodiments

The present disclosure relates to a system for providing a report on a user's vascular health. In this system, a toilet has sensors which detect properties of a user related to the user's cardiovascular and peripheral vascular health and wellness. These properties include those with a known relation to vascular health and properties whose connection has yet to be determined. After collection, the data is compared with preexisting data to generate a report on the user's vascular health. The information in a report can be used for various purposes, including prompting the user to seek additional medical care, providing information to a health or wellness provider, and adding to a database or algorithm for future reference in assessing health and wellness.

A benefit of the disclosed invention is the frequent collection and processing of large amounts of data relative to a user and their health and wellness, including their vascular health. In plain terms, data for the assessment of a user's health and wellness condition can be collected without much deviation from a person's normal use of the toilet; e.g. a person positions themself in front of a toilet, arranges clothing in preparation to use the toilet, stands to urinate or sits to urinate and/or defecate, initiates a flush sequence, and makes themself presentable to leave the privacy of the toilet area. Prior to sitting, a person removes clothing that may be covering bare skin of the legs, thus allowing the bare skin to contact the toilet seat (and sensors contained therein) when the person is seated on the toilet. These actions are relatively ubiquitous in the western world's style of using toilets. The disclosed toilet can include sensors that passively collect health and wellness data, especially vascular health data, because a person performed these actions while using the toilet. Additionally, with slight variations to some user's toilet experience, additional data becomes available. For example, by removing any foot covering (such as socks and shoes) in preparation for using the toilet, additional data can be gathered from the feel and/or ankles. As another example, removal of clothing covering a user's trunk portion can enable data gathering that requires contact with the chest, sides, or back, such as from a stethoscope or ECG leads. As additional examples of variations from a person's normal routine, a person may opt to sit to urinate rather than stand or may opt to put their finger in a pulse oximeter to facilitate data gathering when they otherwise would not opt to do so. This data can then be compared to the user's previous data and/or a data from a relevant population to provide a report of the user's current health and wellness condition, including their vascular condition.

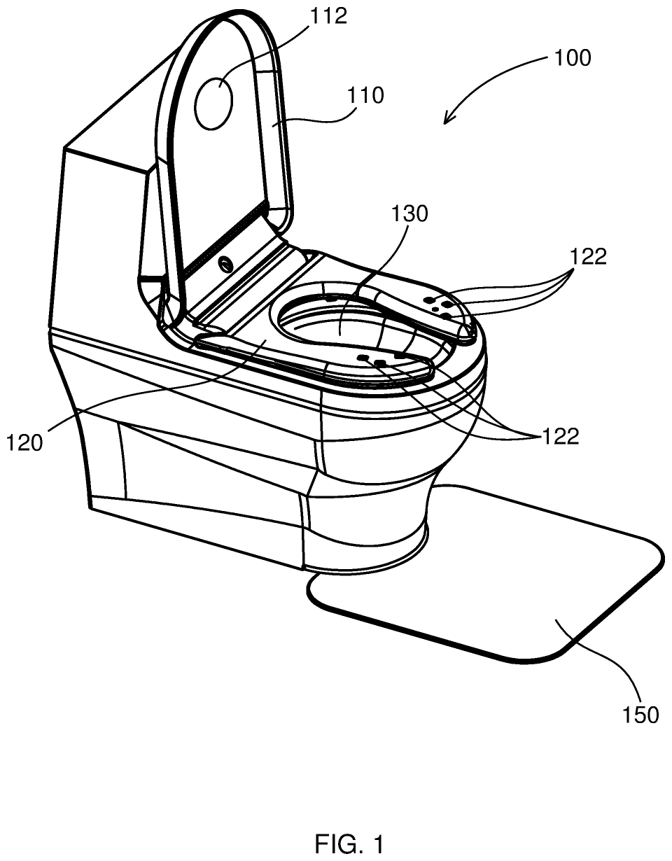

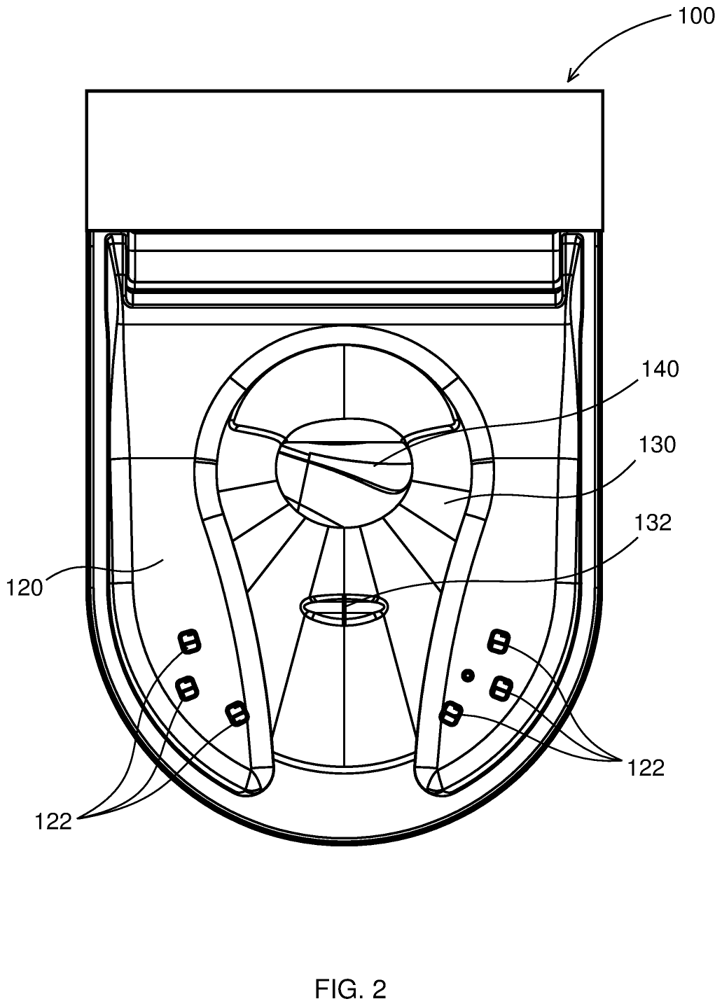

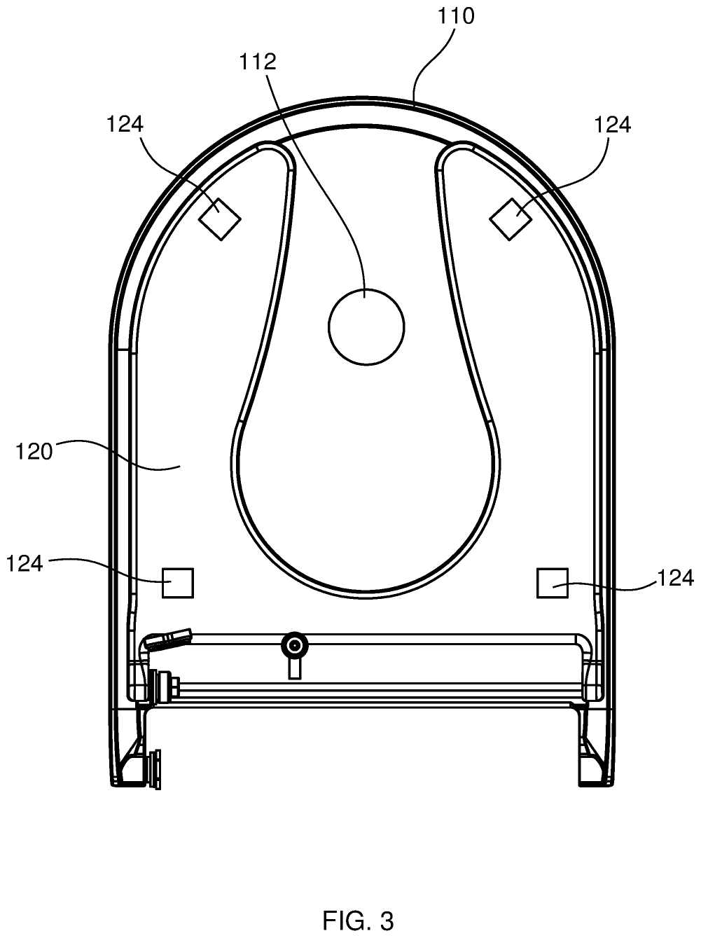

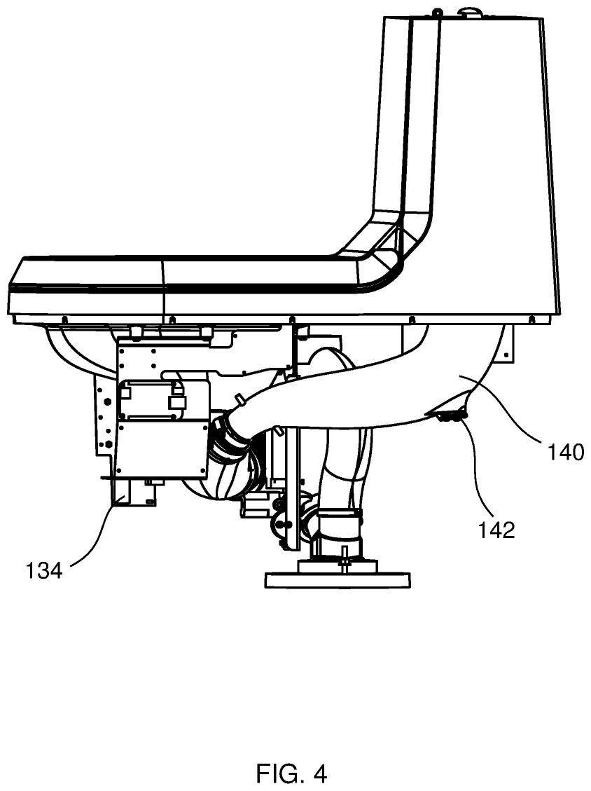

Now referring to FIGS. 1-4, one preferred embodiment of the toilet used in the system is shown. FIG. 1 shows an isometric view of toilet 100 with lid 110 open, showing seat 120 with multiple PPG sensors 122, bowl 130, and foot scale 150. FIG. 2 shows a top view of toilet 100 with lid 110 open, showing seat 120 with multiple PPG sensors 122, bowl 130, and urine volume measure tube 140. Bowl 130 includes urine slit 132, which captures urine for readings spectrometer 134. FIG. 3 is a detail view of the underside of seat 120 with lid 110 behind seat 120. On the underside of seat 120 are weight sensors 124. Shown on lid 110 is stethoscope 112, which includes a microphone for recording audio sounds from a user's trunk portion of the body. FIG. 4 is a detail view showing some of the internal components of toilet 100, including urine volume measure tube 140, urine tube volume sensor 142, and spectrometer 134.

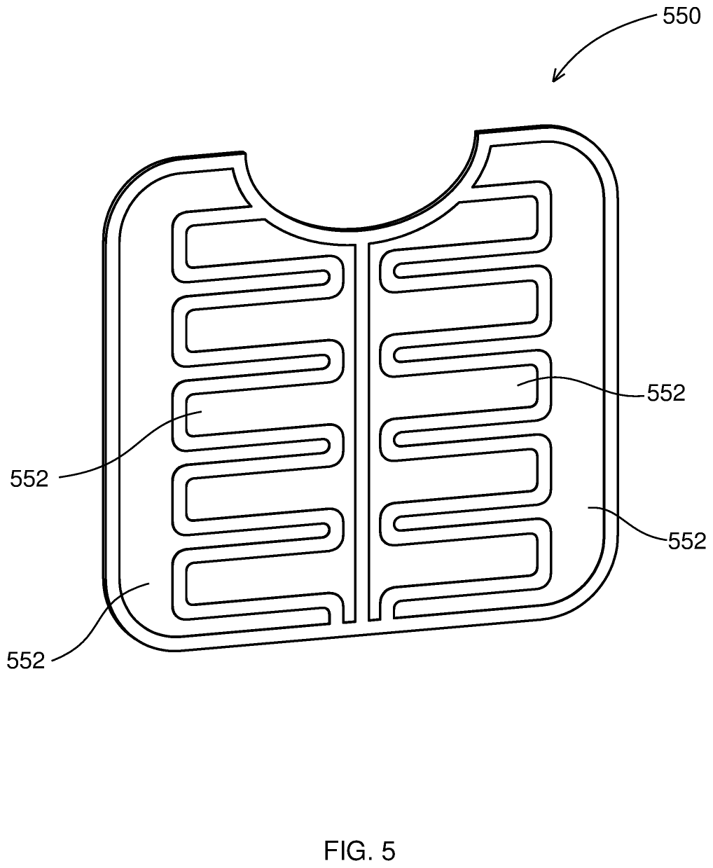

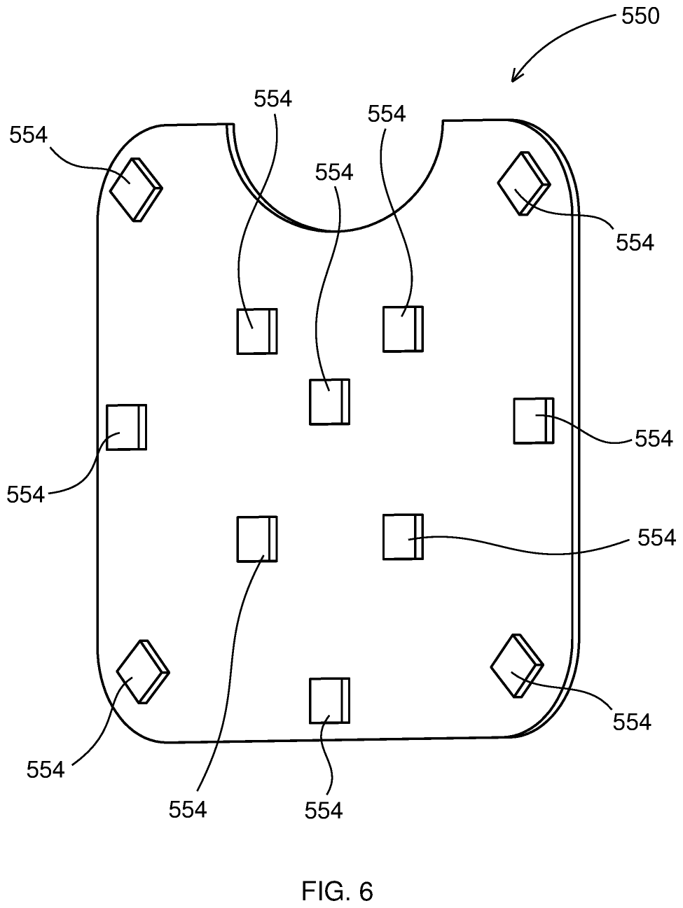

FIGS. 5-6 show one embodiment of a foot pad or foot scale that could accompany a toilet, especially one like that shown in FIGS. 1-4. FIG. 5 shows the top surface of foot scale 550 with bioimpedance sensors 552. A user would place their bare feet on the top surface, contacting two bioimpedance sensors, allowing the user's bioimpedance to be measured. FIG. 6 shows the bottom of foot scale 550 with multiple weight sensors 554 disposed on the bottom surface. One side of weight sensor 554 attaches to the bottom of the scale and the opposing side of weight sensor 554 contacts the ground. When a user places weight on foot scale 550, the various weight sensors 554 cooperate to determine the weight the user is placing on the scale.

In one preferred embodiment, a user walks onto scale 150, and sits down on seat 120, leaving their feet on scale 150. While the user is using the toilet, PPG sensors 122 monitor the user's upper legs; weight sensors 124 monitor the portion of the user's weight on seat 120—including minor, apparent fluctuations that are a result of a user's cardiovascular activity; weight sensors 154 monitor the portion of the user's on foot scale 150, and bioimpedance sensors 152 determine the user's bioimpedance.

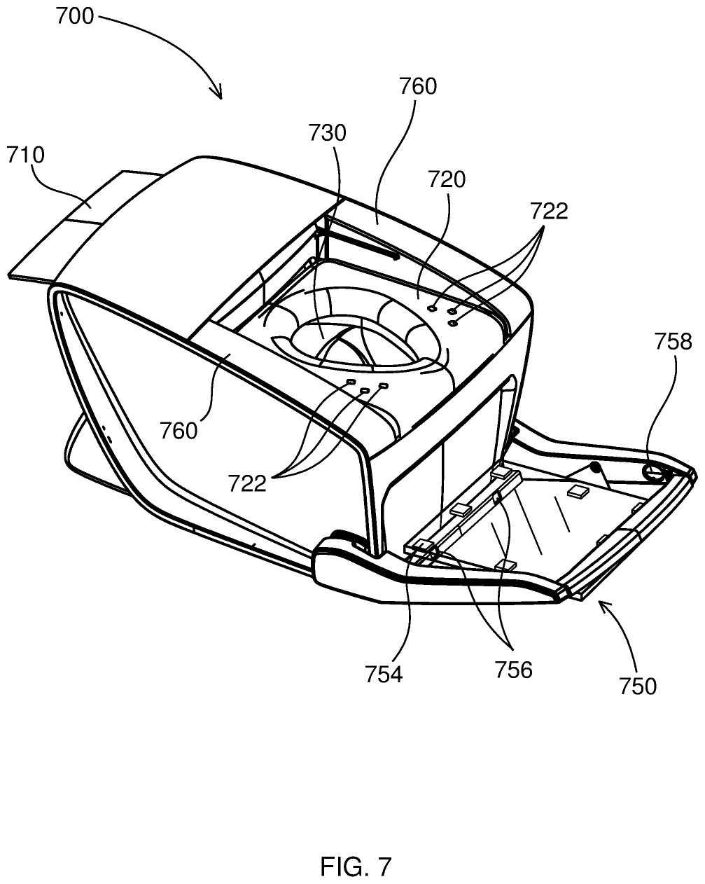

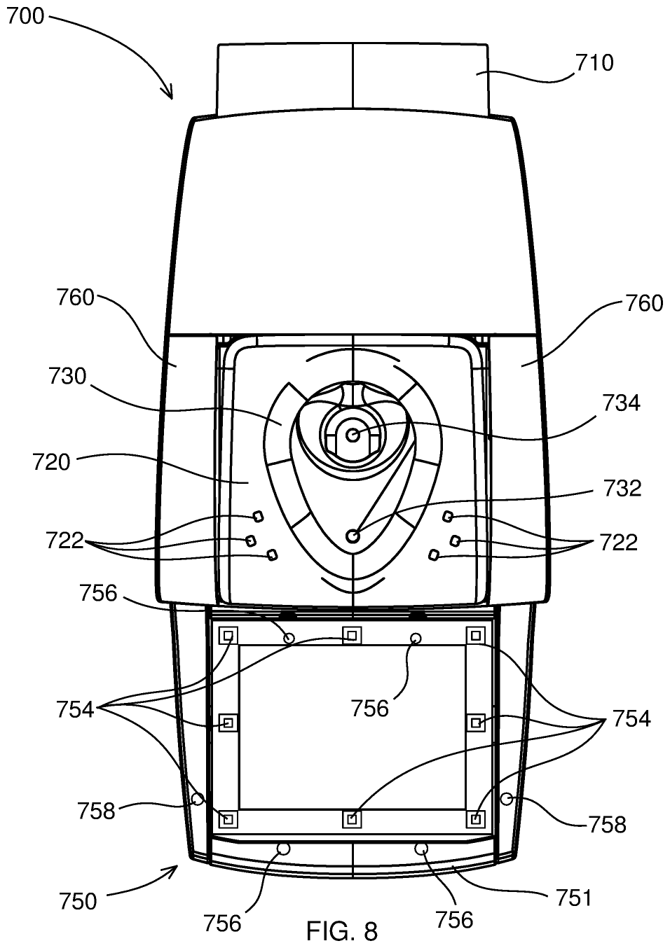

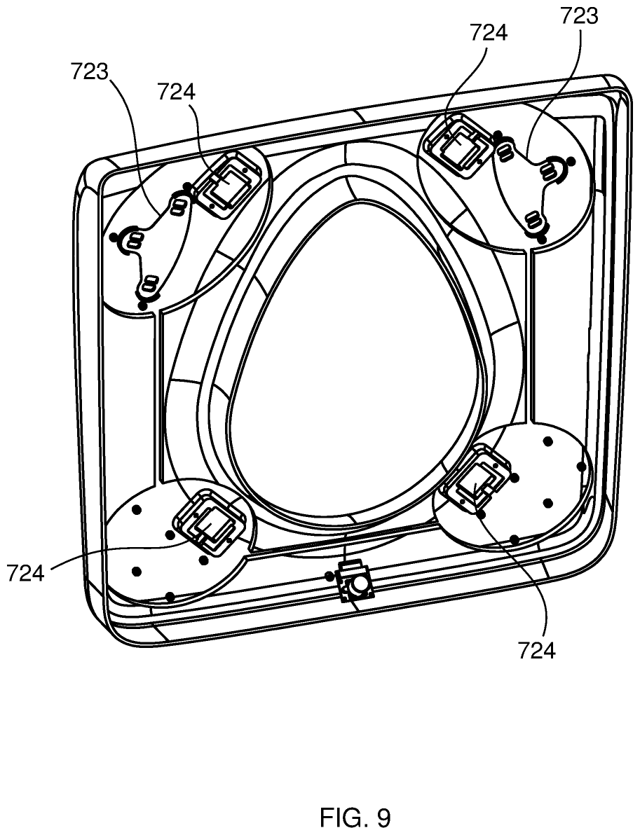

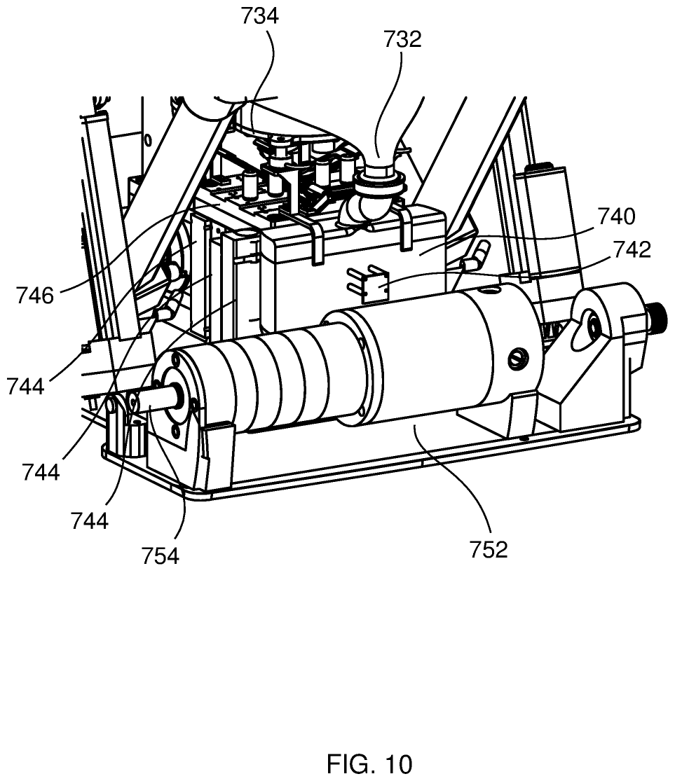

FIGS. 7-10 show another embodiment of the toilet. FIG. 7 shows an isometric view of toilet 700 with lid 710 open, showing seat 720 with multiple PPG sensors 722, bowl 730, foot platform 750, and handles 760. FIG. 8 shows a top view of toilet 700 with lid 710 open, showing seat 720 with multiple PPG sensors 722, bowl 730, foot platform 750, and handles 760. Bowl 730 includes urine receptacle 732 and fecal depository 734. In one preferred embodiment, handles 760 are in a recessed position and can be raised up relative to the toilet. FIG. 9 is a detail view of the underside of seat 720 showing weight sensors 724. FIG. 10 is a detail view showing some of the internal components of toilet 700, including urine receptacle 732, fecal depository 734, urine volume measure chamber 740, urine spectrometer 742, science centers 744, fluid chip receptacle 746, foot platform motor and sensor 752, foot platform motor shaft 753. Foot platform 750 includes frame 751, a glass plate resting on multiple weight sensors 754, foot image sensors 756, and foot IR sensors 758. In one preferred embodiment, science centers 744 and fluid chip receptacle 746 are used in conjunction with excreta analysis, including urine samples and emulsified or otherwise processed excreta.

In one preferred embodiment, a user walks onto platform 750, sits down on seat 720, and platform 750 raises up so the user's feet easily stay on the glass plate. While the user is using the toilet, PPG sensors monitor the user's upper legs; weight sensors 724 monitor the portion of the user's weight on seat 720—including minor, apparent fluctuations that are a result of a user's cardiovascular activity; weight sensors 754 monitor the portion of the user's on foot platform 750, and sensors 754 and 758 monitor the user's feet and lower legs. In one preferred embodiment, sensors 754 and 758 are able to detect properties of the foot, including foot size and shape, coloring, and subdermal vascular properties. These images can undergo image recognition analysis, the results of which can be compared to preexisting data on the same to generate a report on a user's health. Preferably, the report includes information relative to a user's vascular health. Preferably, the comparison is performed by a neural net which has been trained to recognize commonalities to and differences from preexisting images. When the preexisting images are coupled with known health states and/or conditions of the person from whom the images came, the neural net can suggest correlations between the user's images and health states and/or conditions (including neutral or positive ones). Additionally, when the neural net has examined previous data from the same user, the neural net can compare the user's prior state to his or her current state to report on the relative change. Therefore, it may be useful for user data to be averaged, have the mean taken, used in creating trend data, or otherwise be used in creating a baseline against which to compare new user data as it is generated.

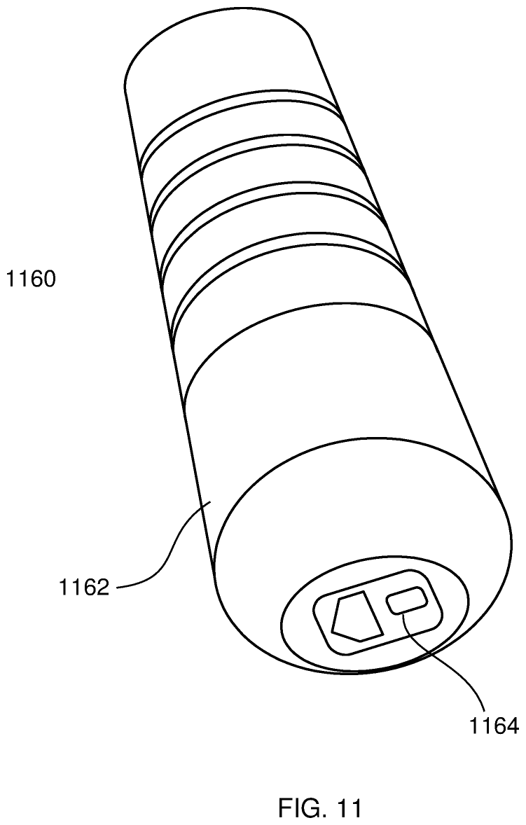

FIG. 11 shows an embodiment of a handle that could accompany a toilet. Handle 1160 includes electrical lead 1162 and PPG sensor 1164. Electrical lead 1162 could be a lead for a bioimpedance sensor and/or an ECG sensor. In one preferred embodiment, a handle would be connected to a cord (with wiring) that connects to the toilet. And another preferred embodiment a handle would be mounted to a structure adjacent to the toilet bowl. In either embodiment a second handle they also do used. A second handle may originate from the same connection point to the toilet or hey location symmetrically opposite or mirrored from the first handle.

In one preferred embodiment, user data, especially data related to vascular health, is compared to preexisting data to create a report on the user's health. More preferably, the report includes information on the user's vascular health. One way of comparing this data is to take cyclical data such as BCG, PPG, or ECG data; perform waveform image recognition analysis on the data; and run the resulting images through a neural net. The neural net will have been previously trained through the input of preexisting data in the form of relevant images paired with known conditions associated with the person who the waveform was generated from represented by the preexisting data (including neutral or healthy conditions). Data and relevant images include those such as BCG, PPG, or ECG waveforms; urine flow results; urine spectrographic results; durometer results; and stethoscopic results. Within the neural net, images of the user's data will be compared to the pre-existing data. The neural net will find similarities between the two sets of data which correlate the users data to conditions the neural net has been trained to recognize and those correlations will be reported back.

There are many methods of assessing vascular condition that relying on the detection of cardiac and cardiovascular-related mechanical motions, including ballistocardiography, phonocardiography, apexcardiography, seismocardiography, kinetocardiography to list just a few. Each of these methods can be completed from a toilet with the necessary structures and sensors.

Take, for example, ballistocardiography. A ballistocardiograph (BCG) is a biological measurement related to cardiovascular and peripheral vascular health. BCG is a measure of the movements of the body resulting from the heart ejecting blood out to the body with each beat. This movement is generally cyclical and occurs with a frequency of about 1 Hz to 20 Hz. BCG results can be largely affected by stiffness of the arteries. A common method for taking this measurement is sensors in contact with the body. Generally, these sensors measure displacement, velocity, and/or acceleration of the body. In one preferred embodiment, BCG measurements are taken from weight sensors located in the toilet seat. These ballistocardiograph sensors can also detect weight or mass from a user with enough resolution to detect minor, momentary, apparent fluctuations in weight resulting from cardiac activity. More preferably, there are 4 weight sensors in the toilet seat. Alternatively, BCG can be monitored using a non-contact method such as an image sensor.

A user's weight or mass, while not necessarily a primary consideration in vascular health, it is also a factor known to be related to vascular health. As noted above, one preferred embodiment of the toilet can detect weight or mass of a user on the seat. However, the seat does not necessary receive all of a user's weight as a user may only sit with a portion of their weight on the seat. There are many ways to determine the total weight of a user, including the use of sensors in the seat, the body of the toilet, handles attached to the toilet, a foot pad or scale on the floor near the toilet, and/or a foot platform extending from the toilet. The selection of where to put sensors and when to gather data from the sensors will be determined by a number of factors, including ease of manufacture, cost, effectiveness, material choices, and other mechanical and electrical design choices for the toilet. In one preferred embodiment, there are 4 sensors in the toilet seat, a sensor each for handle (on either side of the seat), and sensors associate with a foot platform extending from the toilet that can determine weight or mass. This array of sensors can determine weight from sensor readings taken at various times, including when the user is seated on the toilet, when the user is standing on the foot platform, when the user is using the handles, and when a user is transitioning between sitting on the toilet and standing. Alternatively, the foot platform is replaced by a foot pad or scale that sits on the floor and has sensors that allow it to act as a scale. In one preferred embodiment and as noted above, some sensors are used for both BCG and for weight sensing.

Another relevant heart measurement is the electrocardiogram (ECG or EKG), which is a measurement of the electrical activity of the heart using electrodes placed on the skin. Medical uses for this information are varied and often need to be combined with data on the structure of the heart and other physical examination signs in order to provide necessary context for interpreting the ECG data. Conditions for which ECG data is relevant include myocardial infarction (heart attack), cardiac arrhythmias, and medication and anesthesia monitoring. ECG's are generally obtained through the placement of electrodes in contact with a person's skin and detecting the voltage difference between the electrodes. These electrodes may be included in any of the following: the toilet seat, the toilet lid, the toilet base, a foot pad or scale connected to the toilet, a foot platform extending from the toilet, a handle on the toilet, wires attached or which attach to the toilet, integrated into a secondary device which collects the data for comparison with other data from the toilet. In one preferred embodiment, the toilet includes at least 2 electric leads and up to 6 electric leads. Data from the ECG may be used to identify irregularities in the ECG recordings and changes over time.

In one preferred embodiment, the toilet is configured with sensors to help determine pulse wave velocity (PWV). PWV is the velocity at which blood pressure pulse propagates through the circulatory system. It serves as a measure of arterial stiffness and is a useful predictor of future cardiovascular events, “all-cause mortality” independent of conventional cardiovascular risk factors, and even an indicator of target organ damage and hypertension. There are known conditions for which PWV is a relevant indicator. It is also likely that additional conditions will be determined as indicated by PWV, including some conditions for which there is currently no predictor. It is also noteworthy that PWV increases with blood pressure. PWV can be measured in many ways and PWV determination has to take into account the method chosen for gathering data, such as sensor placement on the body. In one preferred embodiment, BCG sensor data is combined with ECG or PPG sensor data and height to determine PWV. A byproduct of determining PWV is pulse transit time (PTT), or the time between when the aortic valve opens and when the resulting blood pulse reaches the sensor.

In one preferred embodiment, the toilet includes a pulse oximeter to detect the percent oxygenation of the user's blood. Percent oxygenation can be obtained in many ways, including absorption and reflection photoplethysmography (PPG) techniques. In absorptive PPG, light is shone through the body (like a fingertip or earlobe) and detected on the other side. In reflective PPG, light is shown onto the body and the reflected light is measured. In either case, a photodiode is used to detect the light, data from which can be used to determine how the light was absorbed by the body. One preferred embodiment includes PPG hardware in the seat of the toilet. Another includes PPG hardware in a finger slit that a user puts their finger into. Additionally, PPG can help determine other properties of a user, such as pulse, blood pressure, autonomic function, and peripheral vascular disease. As noted above, PPG data can be used to determine PWV and PTT.

In one preferred embodiment, the PPG sensors and/or associated circuit boards are optimized for use with the system and/or custom made. Preferably, there are 3 sensors per node. Preferably, there are 3 PPG sensors per circuit board. More preferably, the 3 sensors are within 1″-3″ of each other. Preferably, the sensors user more than one wavelength of electromagnetic radiation (Light). Preferably, one wavelength is 940 nm, which may be termed infrared radiation (IR). Preferably, one wavelength is 660 nm, which may be termed red light (Red). Preferably, one wavelength is 590 nm, which may be termed yellow light (Yellow). Preferably, one wavelength is 520 nm, which may be termed green light (Green). More preferably, the PPG sensor uses 2 of IR, Red, Yellow, and Green. Still more preferably, the PPG sensor uses 3 of IR, Red, Yellow, and Green. Yet more preferably, the PPG sensor uses all 4 of IR, Red, Yellow, and Green. Preferably, IR has a maximum optical power of 1.5 mW/sr. Preferably, Red has a maximum optical power of 2.3 mW/sr. Preferably, Yellow has a maximum optical power of 8.2 mW/sr. Preferably, Green has a maximum optical power of 12.5 mW/sr.

Still regarding the PPG sensor: Preferably, the PPG sensor is closed-loop brightness tunable, which continuously monitors the Light being emitted to make sure each spectrum employed is outputting its target wavelength. Preferably, the DC and AC electrical signals are conditioned independently for simultaneous detection of pulse waveform and multi-wavelength ratios for Spot. Preferably, the sample rate at full load (3 sensors, each using 4 wavelengths with AC and DC operation) is up to 5 kHz. Preferably, the sample rate is 200 microseconds, which requires a relatively low amount of power while still maintaining a high sample rate relative to the speed or biological functions.

In one preferred embodiment, the toilet includes sensors so it can function as a stethoscope. The sounds from the heart typically fall within the range of 10 Hz to 200 Hz and are created from opening and closing of heart valves, blood flow through an orifice, flow of blood into the ventricular chambers, and rubbing of surfaces of the heart. One preferred embodiment of the toilet facilitates stethoscopic measurements by including an acoustic receiver (that can receive the desired range of sounds) to be placed against a user's chest. Alternatively, a secondary device acts as the stethoscope and brings the data together with other data from the toilet, such as a stethoscopic device that wirelessly transmits data to a receiver, a device that plugs into a receiver (like the toilet or a phone), or a phone (wherein the phone is placed proximally to or in contact with the body near the heart). Stethoscopic functionality can enable the toilet to detect and/or record a user's cardia rhythm and heart rate. This data can be used to identify irregular heartbeat (such as may be associated with a heart murmur). This functionality could also detect breathing rate, data from which could be used to identify conditions like congestion or pneumonia.

In some preferred embodiments, there are multiple sets of the same type of sensor that allow for independent detection of one part or side of the body from another. This may result in asymmetric sensor data from a part of the body compared to another part of the body. Not only is there value in comparing one data from one part of the body to historic or preexisting data, but there is value in comparing simultaneously collected asymmetrical data sets to each other. For example, in one preferred embodiment, there are 3 PPG sensors on the left side of the toilet seat and 3 additional PPG sensors on the right side of the toilet seat. Differing data from each side may indicate a number of things, including differences between the user's legs or leg position. As another example, another preferred embodiment includes multiple audio transducer sensors (microphones or stethoscopes) positioned on the toilet. These sensors could listen to the body or functions of the body, have audio recognition analysis run on them, and submit the results to a neural net. Additionally, asymmetric audio transducer data could be used to create a 3-dimentional map of sound producing events.

There are many different qualitative and quantitative biological measurements, including the time a biological process takes, forces associated with the process, acoustic and electrical measurements, visual measurements, and the presence and/or concentration of biomarkers. When taken individually, any one of them may appear to indicate a specific condition, state, or category of conditions or states. As medical understanding grows and the interconnectedness of the body's systems is better understood, biological measurements are increasingly being considered valuable as non-primary indicators related to a person's health and wellness, especially when a group of various measurements are considered collectively. As noted above, a person's weight is considered a non-primary indicator of a person's vascular health.

One benefit of the disclosed toilet is the large variety of biological measurements that can be taken often in a consistent manner, thus creating a large amount of data that can be used in new ways to discover a person's baseline state, how the various measurements represent that state, how that compares to the body's cycles, how that changes with changes in health and wellness (such as getting sick, being exposed to chemical or biological elements, a medical diagnosis or treatment, various stressors a person may encounter), interconnectedness of various measurements where no connection had previously been considered, and a variety of other benefits inherent to having more data available. Increasingly, artificial intelligence (AI) is being implemented to increase the benefit gained from such data. AI can be used in many ways, such as processing the data more quickly, finding correlations within the data, and using those discoveries to provide earlier detections of changes in state of which someone may wish to be aware (such as trending toward a new or different health condition or a likelihood of already having a sickness or disease). One result of which allows for earlier treatment or even preventative care for undesirable conditions where it had not previously been available.

For example, the article “The link between vascular dysfunction, bladder ischemia, and aging bladder disfunction” proposes a link between bladder functions and the vascular system (by Karl-Erik Andersson, Donna B. Boedtkjer, and Axel Forman, published in “Therapeutic Advances in Urology” in 6 Nov. 2016, see https://www.ncbi.nlm.nih.gov/pmc/articles/PMC5167073/). Thus, information gained from a user's single urination event and/or multiple events over time may be helpful in indicating vascular health. Examples of information that can be gathered about a user relative to their urination include specific gravity of the urine, volume of fluid being passed through their system, frequency of urination events, urine flow rate, pressure of urine flow, and the presence of various biomarkers in the urine.

Regarding the above, each has known conditions or issue they are related to. For example, urine specific gravity, volume, and flow rate are indicators of a person's state of hydration. Additionally, urine flow rate is relevant in determining if the prostate is enlarged. Blood in the urine can be indicative of kidney infection or other urinary tract infections. The ratio of albumin to creatinine is relevant to cardiovascular health. The level of sodium, potassium, calcium, chloride, phosphorus, and urea in urine are also relevant biomarkers related to cardiovascular health. Myoglobin and troponin, which are generally only found in the blood and urine if there is sever heart or muscle damage, may be measures of myocardial infarction (also known as “heart attack”). Additionally, examination of urine can identify urinary casts, red and white blood cells, and bacteria in the urine.

Some of the urine related above items are considered primary indicators of cardiovascular or peripheral vascular health, such as myoglobin and troponin levels. Others, such as hydration level, are less obviously related to vascular health, but nevertheless can help create a clearer picture of someone's vascular health. For example, hydration level corelates to stiffnesses within the body. As referenced above, pulse wave velocity is a way to measure arterial stiffness. Additionally, arterial stiffness affects the results of ballistocardiography. Thus hydration level can help improve the understanding gained from PWV and BCG results.

There are many ways of detecting these conditions. For example, a spectrometer can be used to determine the specific gravity of the urine and if there is blood in the urine. Additionally, spectroscopic methods might be used to detect the ratio of albumin and creatinine; myoglobin and troponin levels; as well as the presence or amount of sodium, potassium, calcium, chloride, phosphorus, urea. Spectroscopy may also be used to identify casts, red and white blood cells, and bacteria in the urine. For many of these detection events, the spectrometer detects light which has passed through the urine and can be used to determine the ultraviolet (UV) or visible light (VIS) absorbed by the urine. As another example, a laser or acoustic source and receiver may be used to determine a fluid level in the toilet, which can then be used in combination with data regarding the geometry of the portion of the toilet holding the fluid to determine a urine flow and/or volume from a urination event. One preferred embodiment of the toilet includes a spectrometer which measures light passing through the urine as urine passes through a slit in the toilet. Another preferred embodiment includes a water level in the bowl in fluid connection to a water level internal to piping inside the toilet, which level changes as urine is deposited in the bowl and which level is monitored by the laser or acoustic source and receiver.

Another non-primary indicator that can be used to anticipate or suggest vascular health includes is body fat content. In a publication title “Body Fat Distribution and Risk of Cardiovascular Disease” from 2012 (https://www.ahajournals.org/doi/10.1161/circulationaha.111.067264), obesity rose to be one of the foremost risk factors for cardiovascular disease which can be reduced through lifestyle choices. One preferred embodiment includes bioelectrical impedance detection (or bioimpedance detection), which can be used to estimate body composition, especially body fat and muscle mass. Bioimpedance measures the electrical impedance of body tissues, which can be used to estimate water content in the body. Much of the water in a person's body is stored in muscle, so, when combined with body weight, bioimpedance can be used to determine the body fat content of the body. Additionally, a person's hydration levels can have a large impact on bioimpedance, so any determination of a person's state of hydration can be included to improve the accuracy of body fat content. Thus, bioimpedance data can be used in assessing overall vascular health.

The bioimpedance detection system includes components in direct contact with the skin that generate a voltage and/or detect a voltage. Said a different way, a bioimpedance detection system includes electrodes in contact with the skin, a small electric current passed between the electrodes, and detecting the voltage difference between the electrodes. The impedance of cellular tissue can be modeled as a resistor (representing the extracellular path) in parallel with a resistor and capacitor in series (representing the intracellular path). This results in a change in impedance versus the frequency used in the measurement. Electrodes can be places in a variety of places on the toilet which can contact bare skin, including the toilet seat, a foot pad or foot platform, one or two handles, the base of the toilet, and a backrest. Alternatively, electrodes can be placed in locations of non-direct contact so long as an electrical connection is still possible, such as being in electrical communication with a urine stream or ports that an electrical lead can be plugged into (another end of which is connected to the body). Bioimpedance can provide a body fat estimate based on 2 electrical leads with a current at one electric frequency, but in general, more leads and more frequencies can improve the accuracy of the estimate.

Bioimpedance can be contrasted with other body type or body composition determination systems. As noted above, one simple factor that can be analyzed by one embodiment of the toilet is a person's total weight or mass. Alone, this factor can be helpful in estimating a person's vascular health. However, the accuracy of a vascular health estimate can be greatly improved by considering a person's weight or mass relative to their height to gain a better idea of what that weight is composed of (ie bones, organs, muscles, fat, etc.). For example, body mass index (BMI) is a method for determining body type and is defined as a person's body mass (in kilograms) divided by the square of their height (in meters), resulting in a number expressed in units of kg/(m\hat{ }2). Commonly accepted BMI ranges are underweight (under 18.5 kg/m2), normal weight (18.5 to 25), overweight (25 to 30), and obese (over 30). A BMI can be a determined by an embodiment of the toilet that measures total weight and determines or is given the users height. BMI adds improvements to evaluating vascular health over just looking at weight. However, BMI fails to take into account muscle mass and body fat content, which are both helpful considerations for determining vascular health. While both BMI and bioimpedance can be helpful in determining vascular health, bioimpedance with BMI improves vascular health estimates over using just the BMI or just bioimpedance with weight.

One embodiment of the toilet can be given a value for a user's height, such as a user input or a relevant population average. In an alternative embodiment, the toilet can determine a user's height from sensor data. In another embodiment, the toilet both measures a user's height and compares it to a stored value of a user's height, which could be a historical value from a previous measurement or a value previously provided to the toilet. A person's height can be determined in many ways. One method involves the use of an image or other sensor that detects the relative location of a user's feet and head. For example, the image sensor can provide the amount of viewing angle a person's height takes up. Additionally, an image sensor may have a lens that can focus and, based on the focal distance of the various parts of a user's body, the distances of those body parts can be determined relative to the sensor. The height can then be determined from the data. One sub-method for determining the height involves using trigonometry to calculate the height. Another may simply have users stand at a known location and have heights predetermined based on where the head is located relative to the sensor.

An additional or alternative way to detect body fat and muscle content is through use of a durometer. A durometer can detect the hardness of a user's soft tissue to indicate body fat and muscle tone. In a preferred embodiment, the durometer is placed in direct contact with the skin in a location that generally has a good distribution of fat and muscle. For example, the durometer may be in the seat of the toilet and be applied to the leg. Alternatively, it may be in a lid, backrest, or sides of the toilet and detect the truck portion of a user. Alternatively, it may be in an armrest or body of the toilet and detect the arms. The measurement from the durometer will be affected by the user's hydration level and may therefore become more reliable when combined with hydration level.

Once a sensor has generated data and represented as an electric signal, many things can happen with that data, including it can be transmitted to a controller or processor, processed so it's more usable, analyzed, converted to other units (such as converting a pressure into a weight or converting a fluid height into a volume), stored in long-term or short-term memory, compared with other data (including preferences selected by a person or algorithm), removed from memory, and/or transmitted to a receiver.

Raw sensor data can be directly sent from a sensor to a controller or it can be modified as it is being sent. A digital signal processor (DSP) can be used to modify data in real-time, receiving raw data from a sensor and outputting a modified version of the data. One reason to do this is to make data more usable, such as cleaning up noise. Another reason is improved privacy. The output of the DSP replaces the raw data and the raw data ceases to exist. Therefore, if a sensor generates and outputs data directly to a DSP without storing the data, and the DSP modifies the data sufficiently, then the data cannot be reconstructed into a form which might be a privacy concern for a user. For example, the toilet may include an image sensor capable of generating digital photographs or an acoustic sensor capable of creating a digital audio recording. Relevant health and wellness information may be gleaned from these sensors, but a user of a toilet may not want photographs or an audio recording generated from their use of a toilet. If data from the sensor is passed through a DSP before being sent to a controller, the data can be processed to provide useful information while simultaneously removing elements that might create a privacy concern for a user; in other words, the DSP could process the data so it can no longer be turned into a digital image or audio recording that is recognizable as from a human.

In one preferred embodiment, the toilet has access to read a database of data related to the sensor data. In another embodiment, the toilet could transmit sensor related data to an external location for comparison to an external database. In either case, the toilet may be set up to receive information from a relevant database. Again, in either case, data from a sensor could be compared to a database of relevant health and wellness data and/or could prompt the database to provide additional health and wellness information. This information can be used to additionally estimate a user's health and wellness state, report that state to a user or health and wellness provider, or prompt that the user receive additional assessment or care.

In some preferred embodiments, the toilet provides a user interface to represent information regarding user's health and wellness. Raw or processed data could be directly report, a summary of the data could be reported, a general overview of a user's health and wellness could be provided, or specific aspects of a user's health and wellness could be highlighted. Alternatively, an external device or program may be configured to provide the same, including a computer or mobile device with an app that displays information regarding the user's health and wellness. Alternatively, rather than presenting information about a specific user, a user's health and wellness data could be combined with health and wellness data from other people to create a more generalized understanding of a population.

While the present disclosure often notes embodiments with a sensor or other equipment supporting vascular analysis being located within the toilet, it is possible that some or all of the components are located outside of the toilet. For example, excreta preparation, detection, and processing equipment may be a separate unit adjacent to the toilet which cooperates with the toilet to automatically or semi-automatically receive excreta, prepare a sample of excreta for analysis, test the sample, discard the sample, and prevent cross contamination by cleaning and/or sterilizing portions of the toilet and external equipment that do any portion of the described process. In another example, sensors which detect various user properties may be built into the toilet, the data may be collected and transmitted external to the toilet to a location such as a phone or the cloud where it is analyzed, and results are made available to a user through a phone or web-based application.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the invention.

What is claimed is:

1. A system for providing a report on vascular health of a user comprising:
a toilet with a bowl adapted to receive excreta from the user;
a seat to receive weight from the user wherein the seat comprises:
a weight sensor to detect a portion of the user's weight and apparent fluctuations of the user's weight, which data is used in deriving BCG data for the user;
a PPG sensor to detect PPG data pertaining to vascular properties of the user;
a processor that performs a comparison of the data from the weight sensor and the data from the PPG sensor and preexisting data in a database; and
wherein the processor generates a report on the user's vascular health based on the comparison.

2. The system of claim 1, wherein the report is used in providing the user with information about the user's vascular health or a prompt to seek additional health or wellness care.

3. The system of claim 1, wherein the report is used to prompt a user to seek additional health or wellness care.

4. The system of claim 1, wherein the report is used in providing a heath or wellness provider with information about the user's vascular health.

5. The system of claim 1, wherein the report is electronically stored as a reference for future vascular health comparison.

6. The system of claim 1, wherein the preexisting data is based on data from relevant populations.

7. The system of claim 1, wherein the preexisting data is the derived from the user's previous vascular health data.

8. The system of claim 1, wherein data regarding the user's vascular health is used in calculating an average, mean, or trend to create a baseline about the user's vascular health for that set of data; and the baseline is used as a reference to identify changes of the user relative to the baseline.

9. The system of claim 1, further comprising a second PPG sensor to detect secondary PPG data, wherein the PPG sensor is on the user's left hand side of the seat, the second PPG sensor is on the user's right hand side of the seat, the PPG data and the secondary PPG data are independently collected, and asymmetricallity between the PPG data and secondary PPG data is also used to generate the report.

10. The system of claim 1, wherein waveform image recognition analysis is performed on the BCG data or the PPG data; the processor is part of a neural net; results from waveform image recognition analysis are run through the neural net and compared to the preexisting data; the neural net outputs comparison data relative to the user's relative vascular health; and the processor uses the comparison data in generating the report.

11. The system of claim 1, wherein data from the PPG sensor is used in deriving a blood oxygen level for the user.

12. The system of claim 1, wherein the toilet further comprises a urine sensor.

13. The system of claim 12, wherein the urine sensor monitors the volume of urine deposited in the bowl and is used to derive a user's urine flow.

14. The system of claim 12, wherein the urine sensor monitors the volume of urine deposited in the bowl and is used to derive a user's total urine volume deposited.

15. The system of claim 11, wherein data from the urine sensor is used to detect a ratio of albumin to creatinine in the urine.

16. The system of claim 12, wherein data from the urine sensor is used to detect myoglobin and troponin.

17. The system of claim 12, wherein data from the urine sensor is used to detect in the urine at least one of sodium, potassium, calcium, chloride, phosphorus, and urea.

18. The system of claim 1, further comprising ECG leads to generate ECG data for the user.

19. The system of claim 18, wherein waveform image recognition analysis is performed on the ECG data; the processor is part of a neural net; results from the waveform image recognition analysis are run through the neural net and compared to the preexisting data; the neural net outputs comparison data relative to the user's relative vascular health; and the processor uses the comparison data in generating the report.

20. The system of claim 1, further comprising an additional weight sensor, data from which is combine with data from the seat's weight sensor to create weight data; wherein weight data includes a total weight for the user and is also used to generate the report.

21. The system of claim 1, wherein the height of the user is also used to generate the report.

22. The system of claim 1, wherein BCG data, PPG data, and the height of the user are used in determining a PWV, which is also used to generate the report.

23. The system of claim 1, wherein the user's BMI is also used to generate the report.

24. The system of claim 1, further comprising a bioimpedance sensor that generates bioimpedance data that is useful in determining body composition, and wherein bioimpedance data is also used to generate the report.

25. The system of claim 1, wherein the toilet further comprises a microphone for detecting audio signals relevant to cardiac functions of the user, which signals are also used to generate the report.

26. The system of claim 1, further comprising a durometer to test the user's skin hardness, wherein data from the durometer is also used to generate the report.
